(19) United States
(12) Patent Application Publication
(10) Pub. No.: US 2005/0221434 A1
(43) Pub. Date: Oct. 6, 2005

(54) Receptor of the EDb-fibronectin domains

(76) Inventors: Andreas Menrad, Oranienburg (DE); Alexander Redlitz, Berlin (DE); Marcus Kopplitz, Berlin (DE); Ursula Egner, Berlin (DE); Inke Bahr, Berlin (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/105,475

(22) Filed: Apr. 14, 2005

Related U.S. Application Data
(63) Continuation of application No. 10/676,049, filed on Oct. 2, 2003.
Application No. 10/676,049 is a division of application No. 09/942,117, filed on Aug. 30, 2001, now abandoned.

(30) Foreign Application Priority Data
Sep. 7, 2000 (DE) 10045803.3
May 20, 2001 (DE) 10123/33.4-41

(51) Int. Cl. C07K 14/82; C07H 21/04; C12N 15/09

(52) U.S. Cl. 435/69.1; 435/320.1; 435/325; 530/350; 536/23.5

(57) ABSTRACT

The invention relates to a protein that binds specifically to the ED_{b}-fibronectin domains, process for screening compounds that bind to a receptor of the ED_{b}-fibronectin domains or to ED_{b}-fibronectin domains themselves, as well as the uses of protein.

The invention relates to a protein that binds specifically to the ED_{b}-fibronectin domains.

Fibronectins are an important class of matrix-glycoproteins. Their main role consists in facilitating the adhesion of cells to a number of different extracellular matrices. The presence of fibronectins on the surface of non-transformed cells in culture as well as their absence in the case of transformed cells resulted in the identification of fibronectins as important adhesion proteins. They interact with numerous various other molecules, e.g., collagen, heparan sulfate-proteoglycans and fibrin and thus regulate the cell shape and the creation of the cytoskeleton. In addition, they are responsible for cell migration and cell differentiation during embryogenesis. In addition, they are important for wound healing, in which they make possible the migration of macrophages and other immune cells in the field in question and in the formation of blood clots by making possible the adhesion of blood platelets to damaged regions of the blood vessels.

Fibronectins are dimers of two similar peptides, whereby each chain is approximately 60-70 nm long. At least 20 different fibronectin chains have been identified, of which all are produced by alternative splicing of the RNA-transcript of a single fibronectin gene. An analysis of proteolytic digestion of fibronectin shows that the polypeptides consist of six heavily folded domains of which each domain in turn contains so-called repetition sequences (“repeats”) whose similarities with respect to their amino acid sequence allow a classification in three types (types I, II, III). The central region of both chains of the dimer consists of a section of so-called type-III repetitions, which on average are 90 amino acids long (Kornblihtt, A. R., Viobe-Pedersen, K. and Baralle, F. E., 1983. Isolation and Characterization of cDNA Clones for Human and Bovine Fibronectins. Proc Natl Acad Sci USA, 80, 3218-22). Structural studies have revealed that each type-III repetition consists of seven beta-strands, which are folded into two antiparallel folded sheets, whereby short loop regions are exposed as potential protein-protein-interaction sites (Leahy, D. J.; Hendrickson, W. A.; Aukhil, I. and Erickson, H. P., 1992. Structure of Fibronectin Type III Domain from Tenascin Phased by MAD Analysis of the Selenomethionyl Protein. Science, 258, 987-91). These repetitions of type III make it possible for fibronectins to act as adhesion molecules that interact with cell surface molecules, the so-called “integrins.” The term “integrin” was used for the first time in 1987 in a survey article (Hynes, R. O., 1987, Cell 48, 549-550) to describe a related group of heterodimeric cell surface molecules that act as mediators between the extracellular matrix and the intracellular cytoskeleton and thus induce cell adhesion and migration. These heterodimeric receptors “integrate” or mediate signals from the extracellular environment with specific cellular functions. Up until now, 17 beta-subunits have been known that can interact specifically and non-covalently with more than 20 alpha-subunits, particularly to form as 20 different families (Plow, E. F. et al. 2000, J Biol Chem, 275, 21785-21788). The sequence RGDS, which is found in the tenth repetition of type III of the fibronectin (III-10), in particular mediates the interaction of .fibronectin with at least 8 different integrins. Moreover., it was shown that at least four integrins can interact specifically with fibronectin in an RGDS-independent way (Plow, E. F. et al. 2000, J Biol Chem, 275, 21785-21788). In addition to the III7-, III8-, III9- and III10 sequences, the group of repetition sequences of type III also comprises the repeats EIIIB and EIIIA (ED_{b} and EDa). To date, there has been little or no definition of the functions of these two repetition sequences. A study by Jarnagin, W. et al. (Jarnagin, W.; Rockey, D.; Koteliansky, V.; Wang, S. and Bissell, D. 1994, Expression of Variant Fibronectins in Wound Healing: Cellular Source and Biological Activity of the EIIIA Segment in Rat Hepatic Fibrogenesis. J Cell Biol, 127, 2037-48) suggests that the EDₐ domain is involved in an early response of the liver to an injury and in addition the EDₐ domain seems to be involved in the mediation of cell adhesion processes. A fibronectin isoform, which contains the ED_{b} sequence (ED_{b}-FN or ED-B or EDB), cannot be detected in normal adult tissue, but shows a strong expression in fetal tissue as well as tumor tissue, just as during wound healing.

During the development of a tumor, the extracellular matrix of the tissue in which the tumor grows is modified by proteolytic degradation of already existing matrix components. In this connection, a tumor-induced extracellular matrix is produced that is distinguished from that of normal tissues, offers a more suitable environment for tumor growth, and promotes angiogenesis. Angiogenesis is one of the most important processes in tumor growth and refers to the process in which new vessels stem from existing endothelium-coated vessels. Angiogenesis is a more invasive process that requires a proteolysis of the extracellular matrix, proliferation, directed migration and differentiation of endothelial cells in new capillaries that support the growth of a tumor beyond a certain size.

ED_{b} fibronectin has been associated with the tumor growth. In addition, ED_{b} -FN is concentrated around new blood vessels during angiogenic processes and thus provides a marker for angiogenesis (Castellani, P.; Viale, G.; Dorcaratto, A.; Nicolo, G.; Kaczmarek, J.; Querze, G.; Zardi, L. (1994) Int. J. Cancer 59: 612-618).

The ED_{b} domain is a repetition sequence of type III that comprises 91 amino acids and has an extremely high sequence homology between the rat and chicken fibronectin, which is between 96% and 100%. No RGDS sequences or other amino acid sequences occur within the domains, of which it is known that they mediate an interaction with integrins. The specific function of the ED-B domain is unknown up until now. Three studies have been published that conduct speculations on a general stimulating function with respect to adhesion/cell propagation for various cells.

Chen and Culp (1996), Exp. Cells Res. 223, 9-19 showed that cellular fibronectins contain the ED_{b} domains and adjacent repetition sequences of type II as possibly adhesion-promoting sequences that can be regulated by the cells by alternative splicing of the primary transcript of fibronectin.

In a later study (Chen and Culp, 1998, Clin. Exp. Metast., 16, 1, 30-42), it was possible to show that Ed_{b} induces a cell-signal event that results in a tyrosine phosphorylation of focal adhesion proteins, specifically with a mechanism that is distinguished from the one that is mediated by the repetition sequences III8-9-10, which detect integrins. It is increasingly acknowledged that the cell adhesion to extracellular matrices or to other cells is an important source for a cell signal that is responsible for the regulation of many phenomena, such as, e.g., cell growth, cell differentiation and cell transformation. An adhesion-induced signaling includes the activation of protein-tyrosine-kinases and a cascade of the tyrosine-phosphorylation of different signal-molecules. The authors of the above-mentioned studies would like to point out that for this signal process, the 125 kDa focal adhesion kinase (FAK) is of central importance that links the cell interaction with matrix proteins to the activation of intracellular signal molecules, such as, for example, Src (Xing, Z.; Chen, H. C.; Nowlen, J. K.; Taylor, S. J.; Shalloway, D., and Guan, J. L., 1994, Direct Interaction of v-Src with the Focal Adhesion Kinase Mediated by the Src SH2 Domain. Mol Biol Cell. 5, 413-21), Grb2 (Schlaepfer, D. D.; Hanks, S. K., Hunter, T. and van der Geer, P., 1994, Integrin-Mediated Signal Transduction Linked to Ras Pathway by GRB2 Binding to Focal Adhesion Kinase. Nature, 372, 768-91) and PI-3-kinase (Chen, H. C. and Guan, J. L., 1994, Association of Focal Adhesion Kinase with its Potential Substrate Phosphatidylinositol 3-Kinase. Proc Natl Acad Sci USA, 91, 10148-52). From another Local adhesion protein p130cas, it is also assumed that it is involved in adhesion-mediated signal events and in specific oncogenic activities, although its specific function to date is not explained (Sakai, R.; Iwamatsu, A.; Hirano, N., et al. 1994, A Novel Signaling Molecule, p130, Forms Stable Complexes in Vivo with v-Crk and c-Src in a Tyrosine Phosphorylation-Dependent Manner. EMBO J. 13, 3748-56; Petch, L. A.; Bockholt, S. M., Bouton, A., Parsons, J. T. and Burridge, K., 1995, Adhesion-Induced Tyrosine Phosphorylation of the p130 SRC Substrate. J Cell Sci, 108, 1371-9; Polte, T. R. and Hanks, S. K., 1995, Interaction Between Focal Adhesion Kinase and Crk-Associated Tyrosine Kinase Substrate p130^{Cas}, Proc Natl Acad Sci USA, 92, 10678-82).

The study by Chen and Culp (1998, aaO) shows that the mono-repetition protein ED_{b} was more heavily promoted for the propagation of BALB/c 3T3 cells as well as for inducing FAK-tyrosine phosphorylation than the adjacent repeats III8, etc. The assumption is advanced that in the case of physiological concentrations of cellular fibronectins, the binding of the tetrapeptide RGDS from III10 to the integrins possibly produces a signal of inadequate strength for the cell adhesion, so that no tyrosine-phosphorylation response arises from the interaction between III10 and integrin-mediated mechanisms. It is further assumed that the difference with respect to the response to the various mediated cell adhesions is produced by a varying activation or various small GTP-binding proteins. Three of these proteins—cdc42, rac and rho—which all are members of the ras-superfamily, play important roles in the case of cell-morphological changes. cdc42 acts sequentially upstream from rac and directly induces the appearance of filopodia (Nobes, C. D. and Hall, A., 1995, Rho, rac and cdc42 GTPa-ses Regulate the Assembly of Multimolecular Focal Complexes Associated with Actin Stress Fibers, Lamellipodia and Filopodia, Cell. 81, 53-62). The activation of rac is then responsible for the formation of lamellipodia and the network of actin filaments between the filopodia. Further downstream, rho can be activated by rac and induces focal adhesion and actin stress fibers. All of these events depend on the activation of tyrosine kinase, and it is assumed from FAK that it is involved in these processes. Chen and Culp make the conjecture that the morphological differences between cells that are adherent via 7-ED_{b}-8 as well as cells that are adherent via 8-9-10 are based on the varying activation of the small GTP-binding proteins. The above suggests that an adhesion via 8-9-10 via the integrin-mediated signal path finally leads to an activation of rho to produce focal adhesions and actin stress fibers, while the adhesion of BALB/c-3T3 cells via 7-ED_{b}-8 leads only to an activation of cdc42 proteins and rac proteins, but does not activate rho. For the above-mentioned speculations, however, data are presented in neither of the two studies.

Another study (Hashimoto-Uoshima et al., 1997, J. Cell Sci. 110, 2271-2280) shows that the cell adhesion of cultivated fibroblasts is enhanced by the presence of fibronectin fragments that include the ED_{b}-fibronectin domains. The above suggests that the spliced ED_{b} domain can have an important biological function with respect to enhancing the cell adhesion and cell propagation. The inclusion of EDₐ in fragments in the absence of ED_{b}, however, prevents the formation of good focal adhesions in cells. The authors of this study speculate that this is based on the fact that the inclusion of the two domains in the fibronectin molecule can produce a mechanism with which a cell adhesion is achieved to the extent that strong progressive movement processes are facilitated, in which both adhesion and losses of adhesion are required for strong progressive movement of cells.

Studies on chicken embryos and adult mice showed that ED_{b}-mediated angiogenesis can be blocked by inhibition of the endothelial cell integrin α3β1 (Renato et al., AACR 2001, LB-60).

None of the above-mentioned studies and examinations yield a clear response with respect to the function of the ED_{b} domains, however, and statements are still being made on the identity of a possible receptor (receptors) for the ED_{b} domains.

It is therefore an object of this invention to further clarify the function of the ED_{b} domains. It is another object of this invention to identify a possible specific receptor for the ED_{b} domains. It is another object of this invention to clarify the ED_{b}-specific adhesion mechanism and the interaction with receptor molecules that could be involved in the process of angiogenesis. In addition, it is an object of this invention to identify the ED_{b} region that Is responsible for the specific binding.

This object is achieved by a protein, a) that has the ability to bind specifically to the ED_{b}-fibronectin domains;

b) that is expressed or activated specifically in endothelial cells;

c) that is expressed or activated specifically in the stromal cells of a tumor;

d) that is expressed or activated specifically in tumor cells;

e) whose binding to the ED_{b}-fibronectin domains is inhibited by a polypeptide; and

f) that has an apparent molecular weight of 120-130 kDa for the light chain and 150-160 kDa for the heavy chain, determined by SDS-polyacrylamide gel electrophoresis.

Especially preferred is a protein

a) that has the ability to bind specifically to the ED_{b}-fibronectin domains, whereby the binding region is characterized by at least one sequence that is selected from the group that comprises SEQ ID NOS: 1-3;

b) that is expressed or activated specifically in endothelial cells;

c) that is expressed or activated specifically in stromal cells of a tumor;

d) that is expressed or activated specifically in tumor cells;

e) whose binding to the ED_{b}-fibronectin domains is inhibited by a polypeptide that comprises a sequence that is selected from the group that comprises SEQ ID NOS: 1-3; and

f) that has an apparent molecular weight of 120-130 kDa for the light chain and 150-160 kDa for the heavy chain, determined by SDS-polyacrylamide gel electrophoresis.

Quite especially preferred is a protein,

a) that has the ability to bind specifically to the ED_{b}-fibronectin domains and that comprises the α2β1 chain of the integrin;

b) that is expressed or activated specifically in endothelial cells;

c) that is expressed or activated specifically in stromal cells of a tumor;

d) that is expressed or activated specifically in tumor cells;

e) whose binding to the ED_{b}-fibronectin domains is inhibited by a polypeptide and that comprises the a chain of the integrin; and

f) that has an apparent molecular weight of 120-130 kDa for the light chain and 150-160 kDa for the heavy chain, determined by SDS-polyacrylamide gel electrophoresis.

In a preferred embodiment, the endothelial cells are proliferating endothelial cells.

In a preferred embodiment, the stromal cells are tumor-stromal cells.

In addition, the object is achieved by a protein, whose specific binding to the ED_{b}-fibronectin domains mediates the adhesion of endothelial cells, tumor-stromal cells and tumor cells. The binding region here can be characterized by at least one sequence that is selected from the group that comprises SEQ ID NOS: 1-3 and especially comprises the α2β1 chain of the integrin.

The object is also achieved by a protein whose specific binding to the ED_{b}-fibronectin domains induces the proliferation of endothelial cells. The binding region here can be characterized by at least one sequence that is selected from the group that comprises SEQ ID NOS: 1-3 and especially comprises the α2β1 chain of the integrin.

In addition, the object is achieved by a protein whose specific binding to the ED_{b}-fibronectin domains induces the proliferation, migration and differentiation of endothelial cells in a collagen matrix, whereby the binding region is characterized by at least one sequence. The binding region here can be characterized by at least one sequence that is selected from the group that comprises SEQ ID NOS: 1-3 and especially comprises the α2β1 chain of the integrin.

The object is additionally achieved by a protein that binds to the ED_{b} -fibronectin domains and induces specific signal transduction pathways, whereby at least one gene is induced, for which a protein codes, and which is selected from the group that comprises
focal adhesion kinase,
CD6 ligand (ALCAM),
the α chain of the vitronectin receptor,
the integrated alpha 8 subunit, and
a/the precursor(s) for follistatin-related protein.

The binding region here can be characterized by at least one sequence that is selected from the group that comprises SEQ ID NOS: 1-3 and especially comprises the α2β1 chain of the integrin.

It is preferred that in the induction of specific signal transduction pathways, at least one of the above-mentioned genes is induced at least in one place. In this case, preferably at least one of the above-mentioned genes is induced in two places.

The object is also achieved by an antibody that is able to bind to a protein according to this invention.

In addition, the object is achieved by an antibody that is able to bind to a protein that comprises an amino acid sequence that is selected from the group that comprises SEQ ID NO: 1, SEQ ID NO: 2, SEQ ID NO: 3 and SEQ ID NO:4.

In a preferred embodiment, the antibody is able to inhibit effects that are specific to the ED_{b} domains.

It is preferred that the binding and inhibiting be carried out in vitro and/or in vivo.

In a preferred embodiment, the antibody is monoclonal or recombinant.

In a preferred embodiment, the antibody is an scFv fragment.

The object is also achieved by a cell that expresses a protein according to this invention.

In addition, the object is achieved by a cell that expresses an antibody according to this invention.

In addition, the object is achieved by a phage that expresses an antibody according to this invention.

The object is also achieved by a process for screening with compounds that bind to a receptor of the ED_{b}-fibronectin domains, whereby the process comprises:

Comparison of a response of cells in the presence of one or more of these compounds with the control response of said cells in the absence of these compounds, whereby the cells

express a protein according to this invention or comprise a nucleic acid that codes for this protein, and

whereby the response or the control response is mediated by a receptor of the ED_{b}-fibronectin domains.

In a preferred embodiment, the response or the control response comprises the adherence of cells to surfaces that are coated with the ED_{b}-fibronectin domains or portions thereof.

In a preferred embodiment of the process, a binding region of the ED_{b}- fibronectin domains comprises sequences SEQ ID NOS: 1-4 or portions thereof.

It is preferred that the response or the control response comprise the proliferation of the cells on surfaces that are coated with the ED_{b}-fibronectin domains or portions thereof.

In a preferred embodiment, the response or the control response comprises the proliferation, migration and differentiation of endothelial cells in a collagen matrix, which is used with the ED_{b}-fibronectin domains or portions thereof.

It is preferred that the compounds be selected from the group that comprises antibodies, antibody fragments, artificial antibodies, peptides, low-molecular compounds, aptamers and Spiegelmers.

In a preferred embodiment, the antibodies are recombinant antibodies.

It is preferred that the antibodies be selected from the group that comprises scFv and fragments thereof.

The object is also achieved by a process for screening compounds that bind to the ED_{b}-fibronectin domains, whereby the process comprises:

a) Bringing cells into contact with a fixed concentration of a protein that comprises the ED_{b}-fibronectin domains or a protein with one of the sequences that are represented in SEQ ID NOS: 1-4, in the presence of different concentrations of one or more of the compounds; and

b) Determination of differences in the response of cells to the protein that comprises the ED_{b}-fibronectin domains or a protein with one of the sequences that are represented in SEQ ID NOS: 1-4, in the presence of the compounds in comparison to the control response of cells to the protein that comprises the ED_{b}-fibronectin domains or a protein with one of the sequences that are represented in SEQ ID NOS: 1-4, in the absence of these compounds, whereby
the cells express a protein according to this invention or
comprise a nucleic acid that codes for this protein, and
   whereby the response or the control response is mediated by a receptor of the ED_{b}-fibronectin domains.

In this case, it is preferred that the response or the control response comprise the adherence of the cells to surfaces that are coated with the ED_{b}-fibronectin domains or portions thereof.

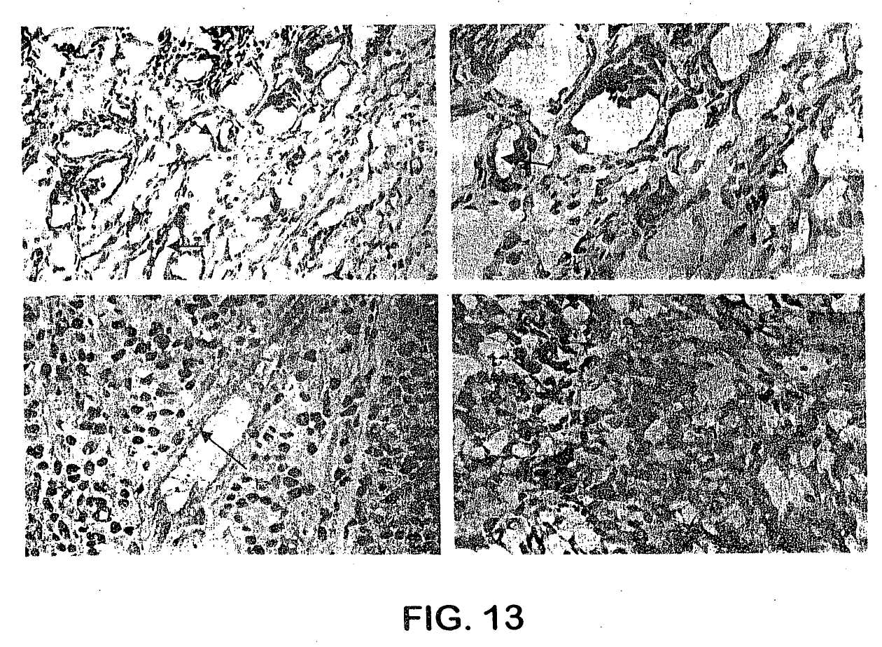

Monoclonal antibodies were produced using standard methods of hybridoma technology and characterized by immunohistology on human tumor-cryosections (see Fig. 13).

By way of example: AK AM-EDBr-2 (murine IgG 1/kappa)

In a preferred embodiment, the response or the control response comprises the proliferation of cells on surfaces that are coated with the ED_{b}-fibronectin domains or portions thereof.

In another preferred embodiment, the response or the control response comprises the proliferation, migration and differentiation of endothelial cells in a collagen matrix, which is mixed with the ED_{b}-fibronectin domains or portions thereof.

It is preferred that the compounds be selected from the group that comprises antibodies, artificial antibodies, antibody fragments, peptides, low-molecular substances, aptamers and mirror aptamers.

The object is achieved in addition by the use of a nucleic acid that codes for a protein that comprises a sequence that is selected from the group that comprises SEQ ID NOS: 1-4 for screening compounds that bind to a receptor of the ED_{b}-fibronectin domains or the ED_{b}-fibronectin domains.

The object is also achieved by the use of a protein according to this invention or an antibody according to this invention for screening compounds that bind to a receptor of the ED_{b}-fibronectin domains or the ED_{b}-fibronectin domains.

The object is also achieved by the use of a cell according to this invention for screening compounds that bind to a receptor of the ED_{b}-fibronectin domains or the ED_{b}-fibronectin domains.

The object is also achieved by the use of a nucleic acid that codes for a protein that comprises a sequence that is selected from the group that comprises SEQ ID NOS: 1-4 to develop antibodies or scFv-fusion proteins for diagnostic or therapeutic purposes.

The object is also achieved by the use of a protein according to this invention to develop antibodies or scFv-fusion proteins for diagnostic or therapeutic purposes. Therapeutic purpose is defined as, i.a., the antiangiogenic treatment with compounds that inhibit the specific interaction between ED_{b} and the receptor. In this connection, the antibodies are directed both against the receptor and against ED_{b}, Whereby the peptides of sequence SEQ ID NOS: 1-3 and stabilized derivatives thereof as well as low-molecular compounds are used.

The object is also achieved by the use of a cell according to this invention to develop antibodies or scF-fusion proteins for diagnostic or therapeutic purposes.

The object is also achieved by the use of a phage according to this invention to develop antibodies or scFv-fusion proteins for diagnostic or therapeutic purposes.

The object is also achieved by the use of a protein that comprises a sequence that is selected from the group that comprises SEQ ID NOS: 1-4 for a pro-angiogenic therapy.

The object is also achieved by the use of a protein that comprises a sequence that is selected from the group that comprises SEQ ID NOS: 1-4 for diagnostic purposes.

The object is also achieved by the use of a protein that comprises a sequence that is selected from the group that comprises SEQ ID NOS: 1-4 in gene therapy.

The object is also achieved by the use of a protein that comprises a sequence that is selected from the group that comprises SEQ ID NOS: 1-4 to coat surfaces to which endothelial cells bind.

In this case, it is preferred that the coating be carried out in vitro or in vivo.

The object is also achieved by the use of a protein that comprises a sequence that is selected from the group that comprises SEQ ID NOS: 1-4 in cell cultures.

The object is also achieved by the use of a protein that comprises a sequence that is selected from the group that comprises SEQ ID NOS: 1-4, together with at least one transplant.

In this case, it is preferred that the transplant be selected from the group that comprises the vessel(s), skin, cornea, kidneys, liver, bone marrow, heart, lungs, bones, thymus gland, small intestine, pancreas, other internal organs as well as portions and cells thereof.

The object is also achieved by the use of a protein that comprises a sequence that is selected from the group that comprises SEQ ID NOS: 1-4, together with at least one implant.

In this case, it is preferred that the implant be selected from the group that comprises lung implants, artificial pacemakers, artificial cardiac valves, vascular implants, endoprostheses, screws, splints, plates, wires, pins, rods, artificial joints, breast implants, artificial cranial plates, false teeth, fillings and bridges.

“Effects that are specific to the ED_{b}-fibronectin domains” are defined as all such effects that are produced by the ED_{b}-fibronectin domains, but not by EIII7, EIII8, etc. Such an effect is described in, for example, Chen et al., 1998 (aaO), i.e., a quick tyrosine-phosphorylation of several intracellular proteins in contrast to the more likely slow phosphorylation after an adhesion mediated by the domains EIII8-9-10. “Low-molecular compounds” are defined as all compounds whose relative molecular mass is below about 1000-1200. “Aptamers” are defined as molecules that are built up to form nucleic acids that are able to act as highly-specific ligands for a large number of biomolecules. “Pro-angiogenic therapy” is defined as any form of therapy in which the angiogenesis is to be required. “Anti-angiogenic treatment/therapy” is defined as any form of treatment/therapy that is designed to inhibit angiogenesis. “Gene therapy” is defined as any form of therapy that is designed to eliminate a gene-related malfunction or the restoration of a normal gene function in the case of diseases, which can be influenced by the elimination or preparation of a protein. It can include the infiltration of foreign DNA into body cells but is not to be considered as limited thereto. “Cell cultures” are to be defined as both cell culture media and cell culture vessels. The cell culture vessels are preferably selected from the group that comprises cell culture bottles, cell culture dishes, cell culture bowls, cell culture plates, microtiter plates, 96-bowl plates, cell culture flasks and bioreactors.

“Diagnostic purposes” are all purposes that serve in the detection of a state of an organism/organ/a cell or the assignment of a current state of an organism/organ/a cell to a specific state category (e.g., a specific disease), for example this can be the use of a kit/chemical reagents/a measuring device, to determine a physical value, such as temperature, etc., or a chemical value, such as concentration, etc., but is not to be considered as limited thereto.

“Therapeutic purposes” are all purposes that serve in the improvement or the healing of a disease state of an organism/organ/a cell. By the phrase “use of a protein together with an implant,” a use that is identical either in time or space is meant. For example, protein molecules can be attached to the implant in its “incorporation” into the body, or else they can be. separated physically from the implant, but they are administered at the same time as the “incorporation” of the implant (injections, etc.).

The invention is now described in detail based on the following examples and figures. Here:

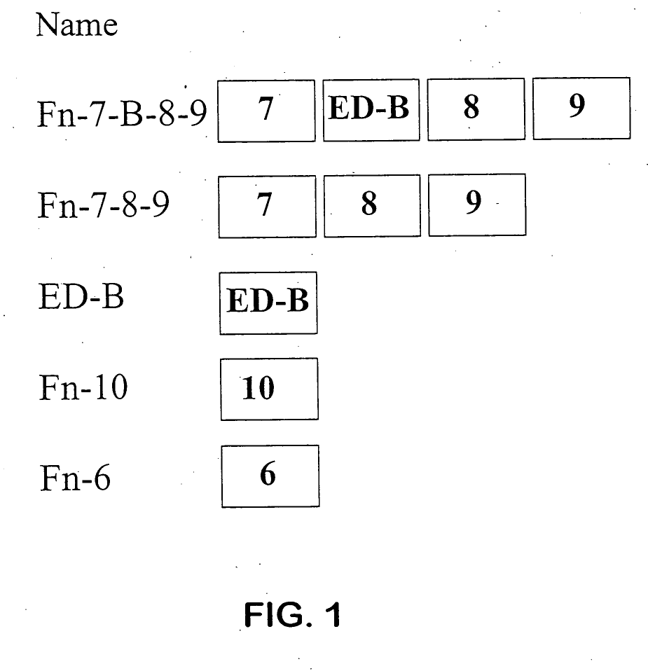

FIG. 1 shows a diagrammatic representation of the repetition sequences of type III that are used in this study;

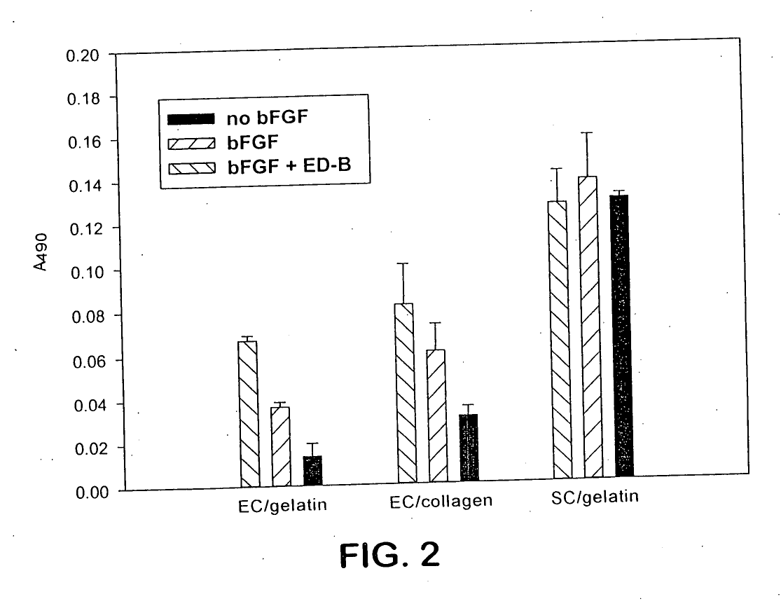

FIG. 2 shows the results of a proliferation assay under the influence of the ED_{b}-fibronectin domains (ED-B) on endothelial cells or human stromal cells on various substrates;

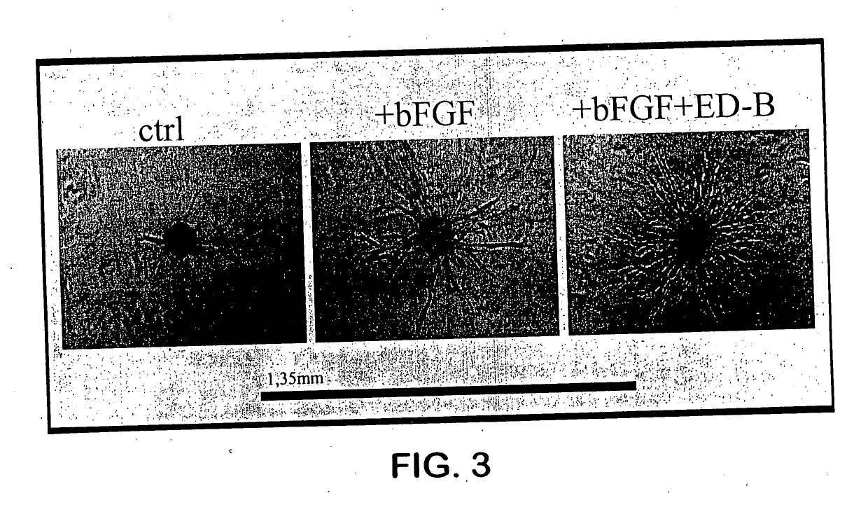

FIG. 3 shows the results of a splintering test (tube formation test) of endothelial cells under the influence of ED-B;

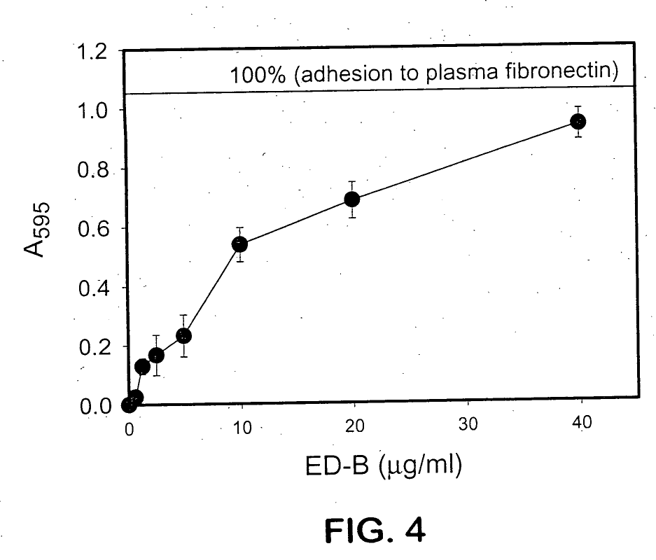

FIG. 4 shows the results of an adherence test, in which the adherence of endothelial cells to surfaces coated with ED-B was tested;

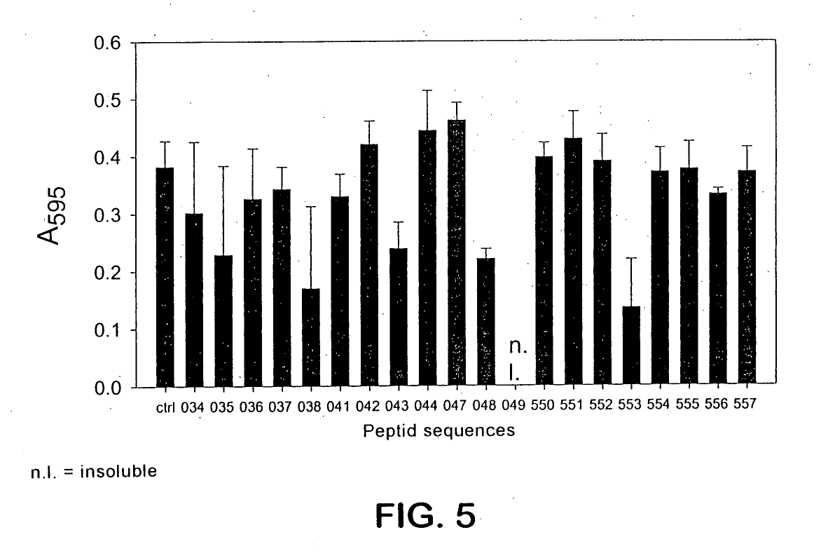

FIG. 5 shows the results of a test, similar to that in FIG. 4, with the exception that the cells were pre-incubated with various synthetic peptides whose sequences are partial sequences of the ED_{b}-fibronectin domains;

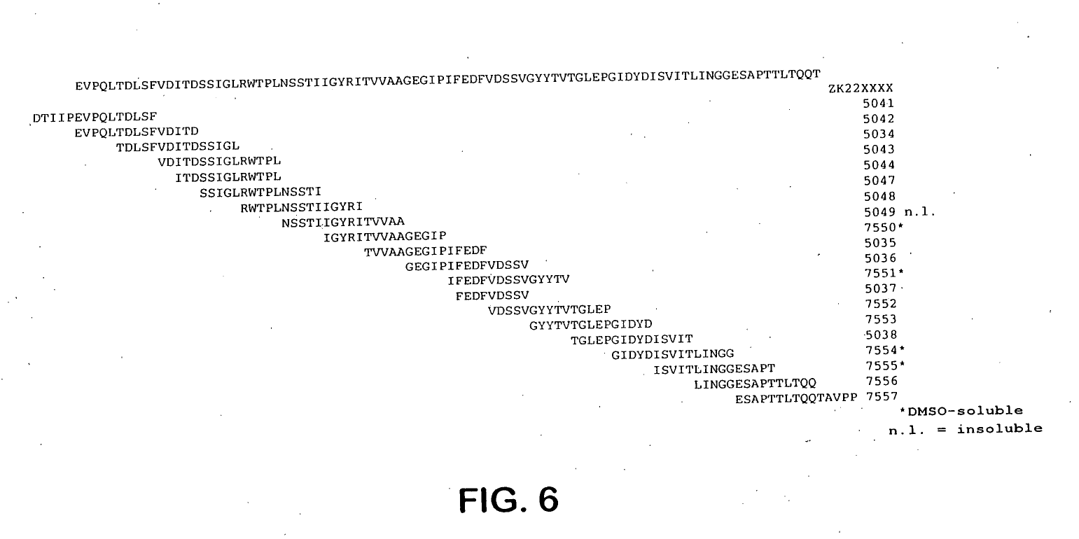

FIG. 6 shows the partial sequences of synthetic peptides from the ED_{b}-fibronectin domains used in FIG. 5;

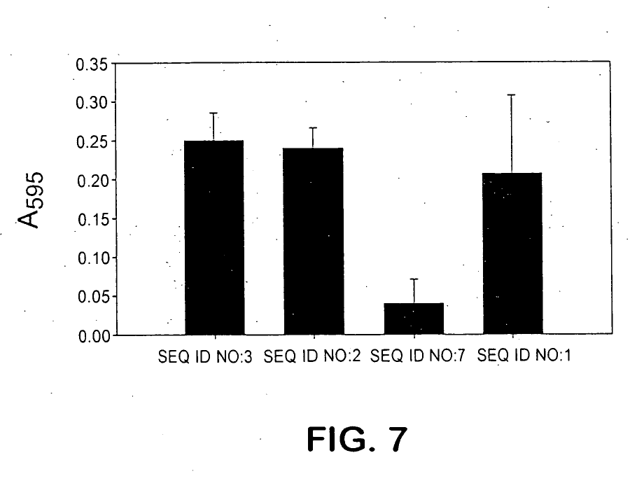

FIG. 7 shows the results of an adherence test of endothelial cells to various synthetic ED-B peptides,

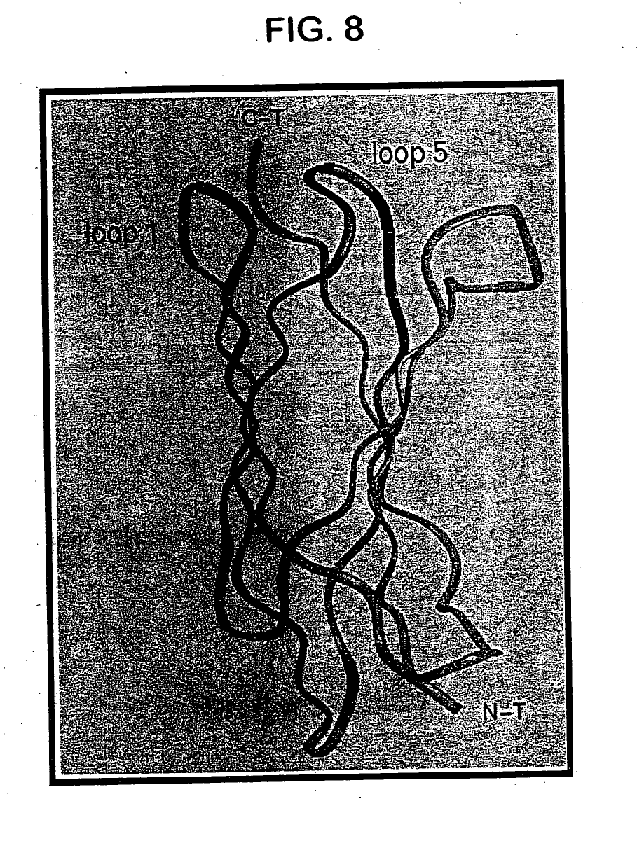

FIG. 8 shows the location of the synthetic peptides found in FIGS. 6-7 in a model structure of the main peptide chain of ED-B;

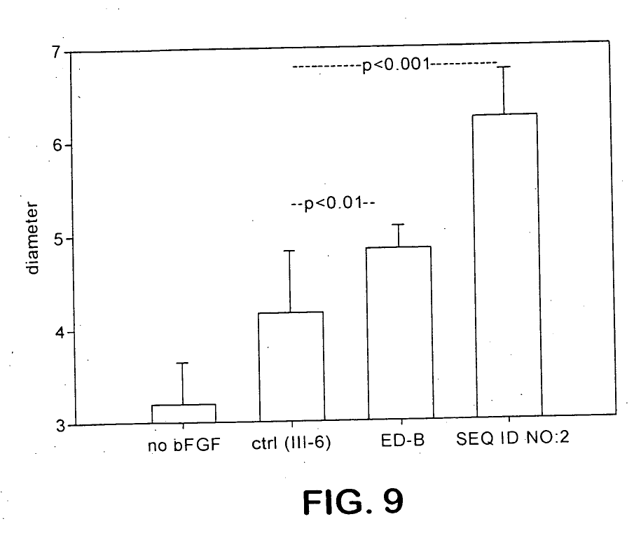

FIG. 9 shows the action of the ED_{b}-fibronectin domains and a peptide derived from loop 5 (SEQ ID NO:2) in the induction of capillary-like structures in a splintering test (tube formation test);

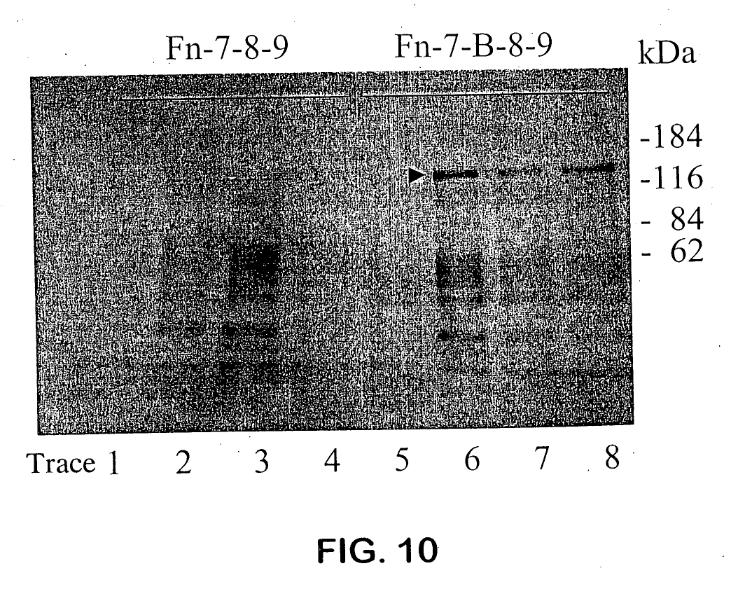

FIG. 10 shows the results of two affinity-chromatography runs with use of Fn-7-8-9 or Fn-7-B-8-9 of cell lysates from surface-labeled human skin-endothelial cells;

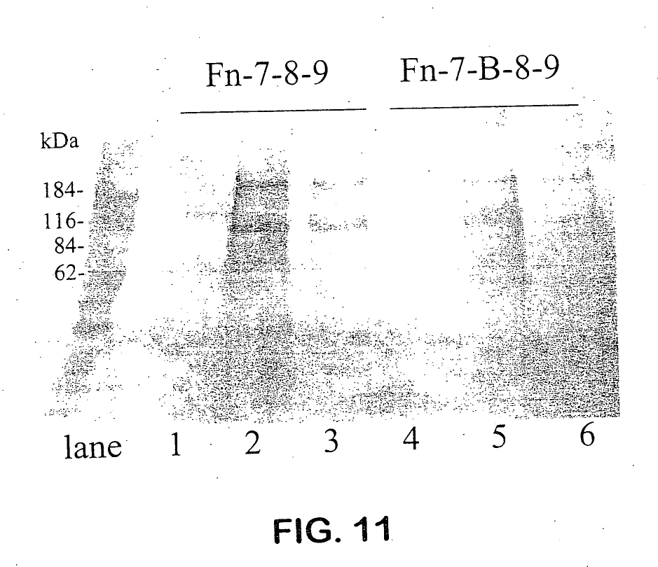

FIG. 11 shows the results of two affinity-chromatography runs with use of Fn-7-8-9 or Fn-7-B-8-9 of cell lysates from surface-labeled human skin-stromal cells;

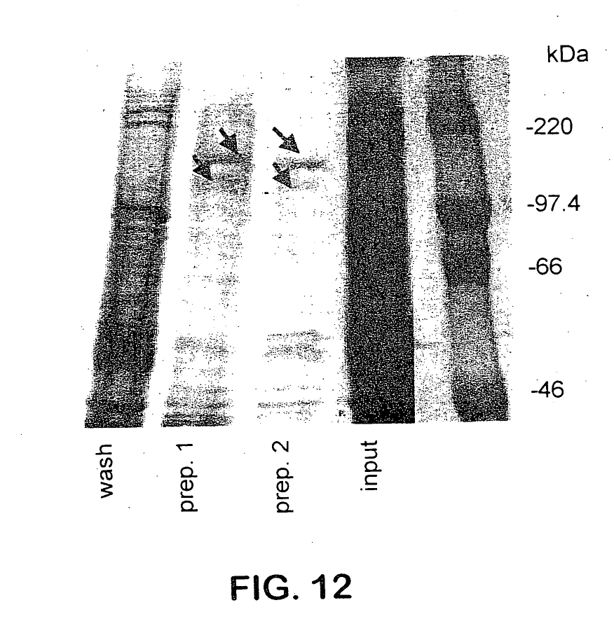

FIG. 12 shows affinity-chromatographic purification of the ED_{b}B receptor;

FIG. 13 shows human tumor cryosections that are characterized-by immunohistology.

FIG. 1 shows various recombinant fibronectin fragments that are used in this study and that have varying domain structures with various repetition sequences of type III. In this case, Fn-7-B-8-9 comprises fibronectin-domains 7, ED_{b} comprises 8 and 9, Fn-7-8-9 comprises domains 7, 8 and 9, ED-B comprises. domains ED_{b}, FN-10 comprises domain 10, and Fn-6 comprises domain 6. These proteins were expressed as proteins provided with an His tag in E. coli and were purified on a nickel-chelate-sepharose column. The number references that are used in this study correspond to those used in the literature. In this case, abbreviations FN-B; ED-B, EDB and ED_{b} all refer to ED_{b}-fibronectin domains in each case and can be viewed as synonymous.

FIG. 2 shows the results of a proliferation assay, in which the action of ED_{b}-fibronectin domains (ED-B) on the proliferation of endothelial cells (EC) or stromal cells (SC) was examined. 1000 cells per bowl were incubated in 96-bowl plates. Soluble ED-B (10 μg/l) was added to the medium during the proliferation assay. After three days, the cell count was determined with the MTS assay. The proliferation of cells was induced by a basic fibronectin growth factor (bFGF). It showed that ED-B had no action in the absence of bFGF, and also no action for the fibronectin domain 10 of type III could be detected in the presence of bFGF in the cells (data not shown). An action of ED-B on human endothelial cell proliferation could be determined in cells that had been flattened out on gelatin (EC/gelatin), also in cells that had been-flattened out on collagen (EC/collagen), whereby the latter effect, however, was not as significant as in the flattening-out on gelatin. In the case of human stromal cells on gelatin (SC/gelatin), even in the absence of bFGF proliferation occurred that considerably exceeded that of human endothelial cells. It could not be increased by the addition of bFGF or bFGF+ED-B. As a yardstick for the cell count, extinction was determined at 490 nm.

For the proliferation assay, the following experimental method was followed:
Material: 96-bowl plate (flat-bottomed), Nunc
Medium: MCDB 131, Pen/Strep, amphotericin (0.25 μg/ml), heparin (20 μg/ml), heat-inactivated FCS (5%)

Method:

Cells, 500-1000 per bowl (96-bowl plate) in 100 μl, are cultivated for 3 days in a medium with bFGF (1-3 ng/ml) or VEGF (30-50 ng/ml). The exact amount should be determined for each batch by titration: the minimum concentration that reaches the maximum proliferation stimulation is optimal. A synchronization of the cells before the experiment is not necessary, but can be done. After 3 days, the cell count is determined with the MTS kit (Promega) according to manufacturer's information. It is recommended to measure the absorption at several points to obtain a maximum absorption in the linear range (0.5; 1; 2; 4 hours).

Controls:

Negative control, no mitogen (no proliferation) (−bFGF/VEGF)
Positive control, with mitogen (maximum stimulation) (+bFGF/VEGF)

FIG. 3 shows the action of ED-B on the splintering of endothelial cells from spheroids. To this end, HUVEC (Human Umbilical Vein Endothelial Cells)—spheroids were embedded in collagens and induced to splinter by the addition of 10 μg/ml of bFGF (basic Fibroblast Growth Factor) in the absence or the presence of 6 μg/ml of ED-B. It was shown that the splintering is induced by the addition of bFGF alone and then could be further stimulated by the addition of ED-B (+bFGF+ED-B).

For the splintering test (tube formation test), the following experimental method was used:

Material:

Methyl cellulose, highest viscosity (Sigma)

Trypsin/EDTA for cell culture (Gibco)

Round-bottom 96-bowl plates (Greiner #650185)

Recombinant bFGF (Gibco #13256-029)

Recombinant VEGF (R & D System)

Anti-rat-CD31 (RDI -#RDI-CD31TLD)

Heparin (Gibco #15077-027)

Solutions:

PBS/Antibiotic agents: cell culture-PBS, 10×Pen/Strep, 2.5 μg/ml of amphotericin

1% gelatin (Difco, autoclaving, and mixing after cooling with Pen/Strep and amphotericin (0.25 μg/ml)
Medium: MCDB 131, glutamine, Pen/Strep, amphotericin (0.25 μg/ml), heparin (20 μg/ml), heat-inactivated FCS (10%)
Growth medium: Medium with 2 ng/ml of bFGF and 10 ng/ml of VEGF

Cells:

HUVEC

Dermal MVEC (passage>4)

Method:

Endothelial cells are dissolved with trypsin/EDTA and diluted with 5000-cells/ml in medium with 0.24% methyl cellulose. 200 μl (1000 cells) each are added to bowls of a Greiner plate and incubated overnight. Round cell clusters (spheroids) are harvested with a 1 ml pipette with beveled tips and centrifuged off. Spheroids are resuspended in 1.2% methyl cellulose/FCS and mixed with neutralized,collagen gel. ED_{b} and bFGF were co-polymerized.

As is evident from the figure, a significant increase in splintering takes place beyond the bFGF-induced value by the addition of ED-B.

FIG. 4 shows the results of an adhesion test of endothelial cells to microtiter-bowl plates, which were coated with ED-B. To this end, endothelial cells were dissolved from their original culture vessel by trypsinization (trypsin/EDTA) of their substrate and then incubated in microtiter-bowl-plates, which were coated with various concentrations (0, 1, 2, 3, 5, 10, 20, 40 μg/ml) of ED-B and left to adhere for one hour. As a negative control, bowls were used that were coated with 1 mg/ml of BSA (bovine serum albumin); the adhesion to BSA (<10%) was subtracted.

The adherence was quantified by staining with crystal violet, followed by a lysis with SDS. The quantification was carried out by measuring the extinction at 595 nm. A line drawn horizontally in the figure at A₅₉₅ nm≈1.06 indicates the 100% adhesion to plasma-fibronectin.

The result of this test indicates that the cells adhere to the surfaces that are coated with ED-B, which suggests a receptor on the cell surface for ED-B.

For the adherence/adhesion test, the following experimental method was used:

Solutions:

1% BSA (Sigma, ethanol-precipitated)

2% serum in PBS (or a trypsin neutralization solution)
Medium: MCDB 131, Pen/Strep, amphotericin (0.25 μg/ml), heparin (20 μg/ml), 0.1% BSA (Sigma, ethanol-precipitated)

0.1% crystal violet, 2% glutaric aldehyde in PBS, sterilized by filtration

2% SDS

Method:

Bowls of a 96-bowl plate (Nunc) are covered with protein for one hour at 37° C. With small proteins (<20 kDa) or peptides, it is recommended to allow the latter to dry on the plate (overnight without a cover under the sterile bank). The bowls are then saturated with 1% BSA for 1 hour at 37° C. Cells are dissolved in 1× trypsin, washed with 2% serum to inactivate the trypsin, and resuspended in medium. If antibodies or peptides are to be tested, the cells are pre-incubated in suspension with the latter for 30 minutes at 37° C. 10⁴ cells per bowl (96-bowl plate) are incubated in a volume of 50-100 μl for 1 hour at 37° C. The supernatant is carefully poured off, the plate can be left inverted to drain on a paper towel for one minute and attached cells are stained with crystal violet/glutaric aldehyde for 15 minutes and attached. The bowls are washed three times with PBS, and the cells are then lysed by adding 2% SDS (15 minutes in the shaker). The absorption at 595 nm is measured. After washing three times with water, the cells, if desired, can be stained again.

Controls:

Negative control: Empty Bowls (BSA control)

Positive control: Plasma-fibronectin (2.5 μg/ml)

% Adhesion=A₅₉₅ (sample): 100×A₅₉₅ (fibronectin)

FIG. 5 shows the results of a test, similar to that of FIG. 4, with the exception that before the adhesion to microtiter-bowl plates coated with ED-B, the endothelial cells were pre-incubated with 250 μM of various synthetic peptides, whose sequence was a partial sequence of the ED_{b}-fibronectin domains. The adherence was determined by the determination of the extinction at 595 nm (A₅₉₅). The peptide designations that are applied in the figure are explained in FIG. 6. In this case, peptide sequence No. 043 corresponds to the sequence that is represented in SEQ ID NO: 1, peptide sequence No. 553 corresponds to SEQ ID NO: 2, peptide sequence No. 038 corresponds to SEQ ID NO: 3. A higher A₅₉₅ value corresponds to a non-inhibited adherence, while a lower A₅₉₅ value corresponds to an inhibition of the adherence by the corresponding peptide.

The method described for FIG. 4 was followed.

FIG. 6 shows the partial sequences of the synthetic ED-B peptides with the selected sequence designations that are removed from the total sequence of the ED_{b}-fibronectin domains. The one-character code for amino acids is used.

FIG. 7 shows the results of a test, similar to that in FIG. 5, except that here the microtiter-bowl plates were not coated with the ED_{b}-fibronectin domains, but rather were pre-incubated with the peptides that have proven inhibitory in the test from FIG. 5, or peptides that have proven not-inhibitory and thus were coated with the latter. In this case, it is shown that the cells in these tests now show adherence in the case of a coating with respectively one of the inhibitory peptides, measured to the A₅₉₅ value, while a peptide from FIG. 5 that has proven not-inhibitory does not lead to any adherence.

The method described for FIG. 4 was followed.

FIG. 8 shows a model structure of the ED_{b}-fibronectin domains (ED-B), from which the locations of inhibitory peptides No. 1 (=SEQ ID NO: 1), No. 2 (=SEQ ID NO: 2) and No. 3 (=SEQ ID NO: 3) are indicated. It shows that these inhibitory peptides are located on loop 1 or loop 5 of the ED-B structure and thus identify the region of the domains via which a binding to the cell or to the receptor that is found on the cell takes place. The model structure of the ED-B domains shown in FIG. 8 is based on an already determined structure of fibronectin domain 7 of type-III. N-T and C-T stand for N- or C-terminus.

FIG. 9 shows the results of a test in which the effect of the addition of ED-B and peptide No. 2, previously determined as inhibitory, as well as the addition of fibronectin domain 6 of type III in the induction of capillary-like structures (tube formation) is studied in the splintering test. It is shown that the maximum effect is produced by the peptide of SEQ ID NO: 2 that inhibits adherence via the basal bFGF-induced penetration into collagen gels. This peptide thus has a stimulating effect on the penetration of endothelial cells in collagen gels. This peptide therefore corresponds to the binding region of ED_{b} and stimulates, analogously to ED_{b} itself, the penetration of endothelial cells in the collagen.

The method described for FIG. 3 was followed.

FIG. 10 shows the results of an affinity chromatography of cell lysate from surface-labeled, human skin endothelial cells. In this respect, proliferating endothelial cells that are biotinylated on the cell surface were lysed with a detergent and subjected to an affinity chromatography, in which short fragments of fibronectin were coupled to sepharose with or without the inserted ED_{b} -fibronectin domains (with the ED_{b}-fibronectin domains=Fn-7-B-8-9, without the ED_{b}-fibronectin domains=Fn-7-8-9). It could be shown that a biontinylated protein with an apparent molecular weight of 120-130 kDa binds specifically to the ED-B-containing fragment (see arrow). The elution is carried out by means of EDTA. Several fractions, described below, were collected. The fractions were then subjected to SDS-PAGE and studied with Western Blot with streptavidin-peroxidase and chemiluminescence (ECL). Traces 1 and 5 show pre-elution fractions, while traces 2, 3, 4 or 6, 7, 8 show the eluted fractions 1, 2 and 3. Traces 1-4 show the chromatography with Fn-7-8-9, while traces 5-8 show the chromatography with Fn-7-B-8-9. The result that is shown here strongly indicates that the prominent band with a molecular weight of between 120-130 kDa is a protein that binds specifically to an ED_{b}-containing fibronectin fragment and thus represents a receptor of the ED_{b}-fibronectin domains.

For the biotinylation and lysis of the endothelial cells, the following experimental method was followed:
Material: Biotinamidohexanoic acid-3-sulfo-N-hydroxysuccinimide-ester; Sigma PBS w/o Mg/Ca (Dulbecco)
   HEPES-buffer: 20 mmol of HEPES, pH 7.6, 1 mmol of CaCl₂, 1 μm of MgCl₂, 0.1% NaN₃, 1% CHAPS (V/V) and Boehringer complete miniprotease inhibitor, EDTA-free, cocktail tablets
      Method: The cell culture bottles are washed respectively 3 times with PBS w/Ca+Mg before and after the biotinylation. Before the last washing process, the biotin buffer (1 mg/15 ml of PBS) is prepared. Into each of the bottles, 5 ml of the buffer (for 225 cm²) or 12.5 ml (500 cm² plates) is pipetted into the center of the bottom, so that the volume can disperse over the entire bottom of the bottle while swinging around. The first culture bottle is then treated with half of the lysis buffer volume. The buffer is also pipetted into the center of the bottom of the bottle and dispersed over the entire surface. The cells are then scraped off with the aid of a cell scraper. The total volume of the first culture bottle is then pipetted into the second bottle, where the process is then repeated. After the last bottle, the volume is transferred into a 50 ml conical centrifuging tube. With the other half of the lysis buffer, this process is repeated in all culture bottles (without cell scrapers) and the final volumes are also added to the centrifuging tubes. It is centrifuged in 50 ml conical cell culture tubes at 3000 rpm, 5 minutes at room temperature (Heraeus table centrifuge). The lysate is pipetted off and ideally should be used immediately for the affinity chromatography (in case of emergency, however, it can also be frozen at −80° C.).

For the covalent coupling of proteins to sepharose, the following process was selected:
Material: Activated CH sepharose 4 B Pharmacia Biotech, Code No. 17-0490-01 1 mmol of HCl, 2.2% NaHCO₃
Method: The HCl is cooled in an ice bath, the sepharose is allowed to heat to room temperature.

Then, the sepharose is washed with 1 mmol of HCl. 10 ml of HCl is required per ml of sepharose. The sepharose is allowed to trickle slowly into the precooled tube, where it then swells for about 15 minutes. (1 g of sepharose corresponds to 3 ml of swollen sepharose.) Then, the tube is centrifuged for 1 minute at 800 U. The supernatant is pipetted off and discarded.

This process is repeated three times.

After the third washing, HCl is again added, the tube is swung around and centrifuged for 3-5 minutes at 800 U. The supernatant is pipetted off, and the pellet is dissolved with 20 ml of millipore water and transferred into two new centrifuging tubes (1 tube each for 7-EDB-8-9 sepharose and for 7-8-9 sepharose, i.e., sepharose to which a polypeptide with repeats III7, ED_{b}, III8 and III9 or III7, III8 and III9 is coupled). The tubes are again centrifuged off immediately, the supernatant is pipetted off, and 1-5 mg of protein/ml of sepharose can be coupled.
(i.e., 2 mg of protein/ml of sepharose 7-8-9 2 mg of protein/ml of 7-EDB-8-9)

The tubes are mixed by being swung around. Then, the addition of 2.2% NaHC3 (50 μl/ml of gel) is quickly carried out. As a result, the residual HCl is neutralized. The tubes are swung around and thoroughly mixed at the maximum stage on a “rocker table” for 1-5 hours.

Then the tubes are centrifuged off again.

To determine the-protein concentration, which is to be used in the covalent coupling to sepharose, a Bradford test was carried out:
Material: BSA stock solution, 2 mg/ml Bradford reagent

Method: The BSA solution is applied as follows to a Nunc-immuno-plate (Maxi Sorp): 5 μg-4 μg-3 μg-2 μg-1 μg (80 μl of Vol.+20 μl of assay)

Pre-dilution for BSA: 5 μg/50 μl=0.1 mg/ml

The stock solution, 2 mg/ml, is diluted by a 1:20 dilution to a concentration of 0.1 mg/ml.

To carry out the affinity chromatography or for elution, the following procedure was selected:

a) Affinity Chromatography
Material: Activated CH sepharose 4B Pharmacia Biotech, Code No. 17-0490-01

Buffer A (20 mmol of HEPES, pH 7.6, 1 mmol of CaCl2, 1 mmol of MgCl2, 0.1% NaN3)

Buffer B (buffer A+150 mmol of NaCl+0.1% Chaps)

Buffer C (buffer A+0.1% Chaps)

PH 4-buffer (millipore water+0.1% glacial acetic acid+0.1% Chaps)

EDTA-buffer (buffer A+200 mmol of EDTA pH 8.5+0.1% Chaps)

Method: The lysate is first put on the column three times.

A tube for collecting the liquid is found below the column. The first 2 ml of the lysate is carefully added to the gel with an Eppendorf pipette. For the additional lysate volume, a measuring pipette is used. It is to be noted that the column is straight. If the column is being used for the first time, a “drying run” with all protein-free buffers is carried out before the actual run. A column charge should be used no more than five times.

If the lysate is frozen (−80° C.), it is first heated in a water bath and then centrifuged (5 minutes at 3000 U).

Fresh lysate, however, is always to be preferred to frozen lysate.

500 μl from the lysate is pipetted off into an Eppendorf vessel.

This is used for the study of the lysate before and after chromatography.

If two columns are used (one each for 7-8-9 sepharose and for 7-B-8-9 sepharose), in each case half of the lysate volume is put on each of the columns. Both columns should have the same flow rate. If this is not the case, the “slower” column is closed for a corresponding length of time. The ideal flow rate is 0.2-0.5 ml/min.

If the lysate has run through the column three times, 500 μl is also pipetted into an Eppendorf vessel from the run, after it was mixed, thus also here a study can be carried out.

Then, 10 column volumes each of buffer B and buffer C are put on the column. The washing process is then completed.

b) Elution
Pre-Elution: Buffer C is put on the column, thus it can be noted whether proteins still remain despite the washing procedure. 500 μl is collected in an Eppendorf vessel. (With two columns corresponding to 2×500 μl).
EDTA-Elution: EDTA complexes the Ca and Mg ions. As a result, the endothelial-cell proteins are eluted, which require Ca and Mg for binding. 2×4 ml of EDTA-buffer is put on the column (or on both columns) and collected in two fractions (El and E2/BE1 and BE2) in Falcon tubes. Then, the tube contents are mixed, and 5000 μl is pipetted off into one (or two) Eppendorf vessel(s).
pH 4-Elution: The actual pH of the buffer is 3.7. Outside of the neutral pH range (pH 6-8), the binding of the receptor to its protein can be inhibited. Also here, as in the EDTA-elution, 2×4 ml of pH 4-buffer is put on the column, collected in two fractions and in each case 500 μl is pipetted off (4.1 and 4.1/B 4.1 and B 4.2).

Then, three column volumes of buffer A are added on the column, so that the acid is washed out. The last acid column remains in the column. The column is closed and kept in the refrigerator.

The 500 μl fractions in the Eppendorf vessels are frozen for at least 15 minutes at −80° C. and then freeze-dried in a “Speed Vac.”

The fractions or pre-elution fractions that are thus obtained were separated with SDS-PAGE and subjected to a Western Blot under reducing conditions.

Fig. 11 shows the same experiment as in FIG. 10, with the exception that here not lysed endothelial cells but rather lysed stromal cells are used. In the Western Blot shown in FIG. 11, traces 1-3 show the elution of an affinity column with Fn-7-8-9, while traces 4-6 show the elution of an affinity column of Fn-7-B-8-9. Traces 1 and 4 are pre-elution factors, while traces 2, 3 or 5, 6 show fractions 1 and 2 of the respective elution run. A prominent band with an apparent molecular weight of 120-130 kDa, as can be seen in FIG. 10, cannot be determined in this cell lysate from human stromal cells.

The features of the invention that are disclosed in the above description, the claims and the drawings can be essential both individually and in any combinations for the implementation of the invention in its various embodiments.

FIG. 12 shows the ED-B binding protein, which was purified by means of affinity chromatography, as described, and was separated by means of SDS-gradient gel electrophoresis (4-12%). The specifically concentrated double bands (arrows) were cut out and analyzed by means of mass spectroscopy.

The sequence analysis clearly identified the isolated protein as the alpha2-beta1-integrin, whereby the predominant heavy band of the betal subunit corresponds to the light band of the alpha2 subunit.

This finding suggests that the binding to EDB is mediated mainly by the betal subunit of the integrin. Corresponding to the cell type examined, other alpha subunits (e.g., alpha2) combined with betal can also mediate the binding to EDB-FN.

FIG. 13 shows human tumor cryosections that are characterized by immunohistology, whereby:
A: Renal cell carcinoma, arrows show the specific staining by means of AK AM-EDBr-2
B: Close-up of the same preparation
C: Hepatocellular carcinoma
D: Melanoma (here no specific staining was found)
   Analysis of the EDB-Receptor

The bands were cut out of a 1D-gel, washed with NH₄HCO₃ solution and acetonitrile, dried, and mixed with trypsin solution for proteolysis of the proteins in gel. The peptides that were eluted from the gel in the digestion solution were concentrated on μC₁₈ columns and desalinated and measured with MALDI-mass spectrometry (=list of peptide masses of the digested protein).

A database search was carried out with the peptide masses found from any gel band. In the case of ambiguous search results, additional MALDI-PSD-spectra (fragment spectra) of an individual peptide were measured. The spectra were used either directly to confirm a suggested peptide sequence (interpretation of the spectrum) or a database search was performed with these spectra.

Bands that were studied:

Band A=Band 1 from preparation 6
   Band 4 from preparation 5
   Band 6 from the acidic elution

Result: Integrin α2
See database search result of Band 4
The spectra from bands 1 and 6 show the same most intense peptides
A PSD-spectrum of a peptide from band 1 confirms a partial sequence of integrin α2
Band B=Band 2 from preparation 6
   Band 5 from preparation 5
   Band 7 from the acidic elution
Result: Integrin β1
   See database search results of bands 5 and 7
   The spectrum of band 2 shows the same most intense peptides
   The database search with a PSD-spectrum from band 2 confirmed Integrin β1
BSA
   Is contained in all three bands

Is confirmed by the database search with a PSD-spectrum and numerous peptide masses

| | |
|---|---|
| | Version 4.10.6 |
| ProFound - Search Result Summary | ©1997-2000 ProteoMetrics |

A:hover { COLOR: red } function togglelt(E1)
{whichlm = event.srcElement;if (E1.style.display ==
“none”){E1.style.display = “”;whichlm.src =
“/prowl/minus.gif;}else{whichlm.src =
”/prowl/plus.gif“;E1.style.display =
”none“;}} A:hover { COLOR: red }
Protein Candidates for search 20010208092948-0121-149234049162
[121056 sequences searched]

| | | Est'd | | | | |
|---|---|---|---|---|---|---|
| Rank | Probability | Z | Protein Information and Sequence Analyse Tools (T) | % | pl | kDa |
| 1 | 1.0e+000 | 2.20 | gi\|4504743\|ref\|NP 002194.1\| | 19 | 5.2 | 129.28 |
| | | | integrin alpha 2 precursor | | | |
| +2 | 2.3e−010 | — | gi\|628012\|pir∥A53933 myosin \| | 15 | 9.6 | 116.17 |
| | | | myr 4 - rat | | | |
| | — | — | gi\|6981242\|ref\|NP 037115.1\| | 15 | 9.6 | 116.12 |
| | | | unconventional myosin from rat 4 for | | | |
| | | | myosin \| heavy chain | | | |
| 3 | 8.3e−011 | — | gi\|7513010\|pir∥T00322 | 15 | 11.5 | 117.58 |
| | | | hypothetical protein KIAA0542 - human | | | |
| 4 | 1.7e−012 | — | gi\|4210973\|gb\|AAD12058.1\| | 11 | 5.9 | 97.99 |
| | | | (AF105016) vacuolar proton translocating | | | |
| | | | ATPase 116-kDa subunit a2 isoform; V-ATPase | | | |
| | | | 116-kDa isoform a2 isoform [Bos taurus] | | | |
| 5 | 5.4e−013 | — | gi\|543747\|so\|P36633\|ABP RAT | 16 | 6.6 | 85.00 |
| | | | AMILORIDE-SENSITIVE AMINE OXIDASE | | | |
| | | | [COPPER-CONTAINING] PRECURSOR (DIAMINE | | | |
| | | | OXIDASE) (DAO) (AMILORIDE-BINDING PROTEIN) | | | |
| | | | (ABP) (HISTAMINASE) | | | |
| 6 | 4.2e−013 | — | gi\|7656867\|ref\|NP 055059.1\| | 12 | 6.8 | 134.71 |
| | | | a disintegrin-like and metalloprotease | | | |
| | | | (reprolysin type) with thrombospondin | | | |
| | | | type 1 motif, 2 | | | |
| 7 | 8.6e−014 | — | gi\|3688530\|emb\|CAA09465.1\| | 11 | 5.8 | 134.87 |
| | | | (AJ011035) phospholipase C beta 2 | | | |
| | | | [Rattus norvegicus] | | | |
| +8 | 6.5e−014 | — | gi\|4504085\|ref\|NP 000399.1\| | 21 | 7.0 | 80.80 |
| | | | glycerol-3-phosphate dehydrogenase 2 | | | |
| | | | (mitochondrial) | | | |
| | — | — | gi\|7446012\|pir∥G02093 | 21 | 7.3 | 80.82 |
| | | | glycerol-3-phosphate dehydrogenase - human | | | |
| 9 | 5.0e−014 | — | gi\|7513725\|pir∥T29098 | 14 | 8.1 | 114.87 |
| | | | microtubule-associated protein 4, muscle- | | | |
| | | | specific - mouse (fragment) | | | |
| 10 | 4.7e−014 | — | gi\|6005970\|ref\|NP 009078.1\| | 22 | 9.6 | 81.59 |
| | | | zinc finger protein 175 | | | |

Input Summary

| | |
|---|---|
| Date & Time | Thu Feb 08 08:29:55 2001 UTC (Search Time: 6.30 sec.) |
| Sample ID | EDB Fibronektin, Bande 4 |
| Database | NCBInr [..\databases\nr] |
| Taxonomy Category | Mammalia (mammals) |
| Protein | 80-135 kDa |
| Mass Range | |
| Protein pl Range | 0.0-14.0 |
| Search for | Single protein only |
| Digest Chemistry | Trypsin |
| Max Missed Cut | 2 |
| Modifications | +C3H5ON@C(Partial); +O@M(Partial); |
| Charge State | MH+ |
| Peptide Masses | |
| (Da, Average) | |
| Tolerance(AVG) | 1.00 ppm |
| | 935.536 1007.504 1179.635 1222.729 1277.731 1307.689 1473.816 1479.833 |
| Peptide Masses | 1510.835 1553.895 1567.768 1586.801 1638.888 1707.772 1819.830 |
| (Da, Monoisotopic) | 1851.993 1915.959 1931.980 1947.990 1973.966 1993.998 2044.968 |
| | 2051.077 2068.095 2095.065 2150.093 2224.097 2283.137 2344.115 |
| | 2501.214 2705.123 2775.304 2872.336 2902.333 2932.502 3052.424 |
| | 3280.542 |
| Tolerance(MON) | 50.00 ppm |
| Number of | 37 |
| Peptides | |

ProteoMetrics' ProFound is based on ProFound at The Rockefeller University
[search + transmission time: >=6.33 sec]
function expandlt(whichE1) {whichE1.style.display =
(whichE1.style.display == “none”)? “”: “none”;}

| | |
|---|---|
| | Version 4.10.6 |
| ProFound - Search Result Summary | ©1997-2000 ProteoMetrics |

A:hover{ COLOR: red } function togglelt(E1)
{whichlm = event.srcElement;if (E1.style.display ==
“none”){E1.style.display = “”;whichlm.src =
“/prowl/minus.gif”;}else{whichlm.src =
“/prowl/plus.gif”;E1.style.display = “none”;
}} A:hover { COLOR: red }
Protein Candidates for search 20010207110038-0035-149234049162
[121056 sequences searched]

| | | Est'd | | | | |
|---|---|---|---|---|---|---|
| Rank | Probability | Z | Protein Information and Sequence Analyse Tools (T) | % | pl | kDa |
| +1 | 1.0e+000 | 1.15 | gi\|124963\|sp\|P05556\|ITB1 | 17 | 5.3 | 88.45 |
| | | | HUMAN FIBRONECTIN RECEPTOR BETA SUBUNIT | | | |
| | | | PRECURSOR (INTEGRIN BETA-1) (CD29) | | | |
| | | | (INTEGRIN VLA-4 BETA SUBUNIT) | | | |
| | — | — | gi\|762977\|emb\|CAA33272.1\| | 11 | 5.8 | 88.18 |
| | | | (X15202) Fn receptor beta prechain | | | |
| | | | [Mus musculus] | | | |
| | — | — | gi\|72070\|pir∥IJMSFB | 11 | 5.8 | 88.31 |
| | | | fibronectin receptor beta chain precursor - mouse | | | |
| | — | — | gi\|8393636\|ref\|NP 058718.1\| | 11 | 5.8 | 88.48 |
| | | | integrin, beta 1 | | | |
| | — | — | gi\|124964\|sp\|P09055\|ITB1 | 11 | 5.7 | 88.21 |
| | | | MOUSE FIBRONECTIN RECEPTOR | | | |
| | | | BETA SUBUNIT PRECURSOR (INTEGRIN BETA-1) | | | |
| | — | — | gi\|10336839\|qb\|AAG16767.1\|AF192528 1 | 11 | 5.3 | 88.25 |
| | | | (AF192528) integrin beta-1 subunit | | | |
| | | | [Sus scrofa] | | | |
| | — | — | gi\|1708573\|sp\|P53712\|ITB1 | 9 | 5.3 | 85.31 |
| | | | BOVIN FIBRONECTIN RECEPTOR BETA SUBUNIT | | | |
| | | | (INTEGRIN BETA-1) (CD29) (INTEGRIN VLA-4 BETA | | | |
| | | | SUBUNIT) | | | |
| | — | — | gi\|1708574\|Sp\|P53713\|ITB1 | 9 | 5.2 | 88.08 |
| | | | FELCA FIBRONECTIN RECEPTOR BETA SUBUNIT | | | |
| | | | PRECURSOR (INTEGRIN BETA-1) (CD29) | | | |
| | | | (INTEGRIN VLA-4 BETA SUBUNIT) | | | |
| 2 | 1.9e−004 | — | gi\|5453910\|ref\|NP 006216.1\| | 8 | 6.2 | 85.75 |
| | | | phospholipase C. delta 1 | | | |
| +3 | 7.7e−005 | — | gi\|1589134\|prf∥22103.13A | 10 | 5.9 | 83.46 |
| | | | phosphatidylinositol 3- | | | |
| | — | — | kinase:SUBUNIT=55 kD regulatory | | | |
| | | | [Rattus norvegicus] | | | |
| | — | — | gi\|6981358\|ref\|NP 037137.1\| | 8 | 5.9 | 83.51 |
| | | | phosphoinositide 3-kinase p85 (other | | | |
| | | | splicing variants: p55 and p50) | | | |
| 4 | 1.8e−005 | — | gi\|1163174\|gb\|AAA85505.1\| | 8 | 5.8 | 86.48 |
| | | | (U32575) similar to yeast Sec6p. Swiss- | | | |
| | | | Prot Accession Number P32844; similar to | | | |
| | | | mammalian B94, Swiss-Prot Accession Number | | | |
| | | | Q03169; Method: conceptual translation supplied | | | |
| | | | by author [Rattus norvegicus] | | | |
| 5 | 1.1e−005 | — | gi\|2137061\|pir∥PC4183 | 10 | 5.9 | 84.62 |
| | | | 1-phosphatidylinositol phosphodiesterase (EC | | | |
| | | | 3.1.4.10) delta 1 - Chinese hamster (fragment) | | | |
| 6 | 6.1e−006 | — | gi\|9910238\|ref\|NP 064388.1\| | 10 | 9.6 | 93.37 |
| | | | general control of amino acid synthesis, | | | |
| | | | yeast homolog-like 2 | | | |
| 7 | 2.4e−006 | — | gi\|10047327\|dbi\|BAB13451.1\| | 6 | 9.0 | 97.20 |
| | | | (AB046845)KIAA1625 protein [Homo sapiens] | | | |
| 8 | 1.1e−006 | — | gi\|5032191\|ref\|NP 005793.1\| | 10 | 9.7 | 93.48 |
| | | | tumor protein p53-binding protein | | | |
| +9 | 9.9e−007 | — | gi\|9910260\|ref\|NP 064581.1\| | 9 | 8.7 | 98.86 |
| | | | HCNP protein | | | |
| | — | — | gi\|6330235\|dbi\|BAA86491.1\| | 6 | 5.6 | 87.80 |
| | | | (AB033003) KIAA1177 protein [Homo sapiens] | | | |
| +10 | 9.6e−007 | — | gi\|9453796\|emb\|CAB99365.1\| | 11 | 6.8 | 96.83 |
| | | | (AL117378) dJ131F15.2 (phosohodiesterase | | | |
| | | | I/nucleotide pyrophosphatase 1 (homologous | | | |
| | | | to mouse Ly-41 antigen) (PC1, NPPS)) | | | |
| | | | [Homo sapiens] | | | |
| | — | — | gi\|129678\|sp\|P22413\|PC1 | 7 | 6.8 | 99.91 |
| | | | HUMAN PLASMA-CELL MEMBRANE GLYCOPROTEIN | | | |
| | | | PC-1 [INCLUDES: ALKALINE PHOSPHODIESTERASE | | | |
| | | | I; NUCLEOTIDE PYROPHOSPHATASE (NPPASE)] | | | |

Input Summary

| | |
|---|---|
| Date & Time | Wed Feb 07 10:00:44 2001 UTC (Search Time: 5.91 sec.) |
| Sample ID | EDB Fibronektin, #0824, Bande 5 |
| Database | NCBInr [..\databases\nr] |
| Taxonomy Category | Mammalia (mammals) |
| Protein | 80-100 kDa |
| Mass Range | |
| Protein pl Range | 0.0-14.0 |
| Search for | Single protein only |
| Digest Chemistry | Trypsin |
| Max Missed Cut | 2 |
| Modifications | +C3H5ON@C(Partial); +O@M(Partial); |
| Charge State | MH+ |
| Peptide Masses | |
| (Da, Average) | |
| Tolerance(AVG) | 1.00 ppm |
| | 881.288 927.495 983.498 1007.525 1222.666 1376.820 1422.642 1439.854 |
| Peptide Masses | 1475.797 1479.791 1553.852 1567.742 1638.888 1781.886 1915.892 |
| (Da, Monoisotopic) | 1961.078 2019.135 2044.949 2225.083 2283.131 2470.203 3143.411 |
| | 3299.415 3323.912 3337.675 |
| Tolerance(MON) | 50.00 ppm |
| Number of Peptides | 25 |

proteoMetrics' ProFound is based on ProFound at The Rockefeller
University [search + transmission time: >=5.94 sec]
function expandlt(whichE1) {whichE1.style.display =
(whichE1.style.display == “none”)?
“”:“none”;}

| | |
|---|---|
| | Version 4.10.6 |
| ProFound - Search Result Summary | ©1997-2000 ProteoMetrics |

A:hover { COLOR: red } function togglelt(E1)
{whichlm = event.srcElement;if (E1.style.display ==
“none”){E1.style.display =“”;whichlm.src =
“/prowl/minus.gif;}else{whichlm.src =
”/prowl/plus.gif';E1.style.display = “none”;}}
A:hover { COLOR: red }
Protein Candidates for search 20010207110746-00D6-149234049162
[121056 sequences searched]

| Rank | Probability | Est'dZ | Protein Information and Sequence Analyse Tools (T) | % | pl | kDa |
|---|---|---|---|---|---|---|
| +1 | 1.0e+000 | 1.61 | gi\|124963\|sp\|P05556\|ITB1 | 18 | 5.3 | 88.45 |
| | | | HUMAN FIBRONECTIN RECEPTOR BETA SUBUNIT | | | |
| | | | PRECURSOR (INTEGRIN BETA-1) (CD29) | | | |
| | | | (INTEGRIN VLA-4 BETA SUBUNIT) | | | |
| | — | — | gi\|10336839\|gb\|AAG16767.1\|AF192528 1 | 12 | 5.3 | 88.25 |
| | | | (AF192528) integrin beta-1 subunit | | | |
| | | | [Sus scrofa] | | | |
| | — | — | gi\|762977\|emb\|CAA33272.1\| | 12 | 5.8 | 88.18 |
| | | | (X15202) Fn receptor beta prechain | | | |
| | | | [Mus musculus] | | | |
| | — | — | gi\|72070\|pir∥IJMSFB | 12 | 5.8 | 88.31 |
| | | | fibronectin receptor beta chain precursor- mouse | | | |
| | — | — | gi\|124964\|sp\|P09055\|ITB1 | 12 | 5.7 | 88.21 |
| | | | MOUSE FIBRONECTIN RECEPTOR BETA SUBUNIT | | | |
| | | | PRECURSOR (INTEGRIN BETA-1) | | | |
| | — | — | gi\|8393636\|ref\|NP 058718.1\| | 12 | 5.8 | 88.48 |
| | | | integrin, beta 1 | | | |
| | — | — | gi\|1708573\|sp\|P53712\|ITB1 | 10 | 5.3 | 85.31 |
| | | | BOVIN FIBRONECTIN RECEPTOR BETA SUBUNIT | | | |
| | | | (INTEGRIN BETA-1) (CD29) (INTEGRIN VLA-4 BETA | | | |
| | | | SUBUNIT) | | | |
| | — | — | gi\|1708574\|sp\|P53713\|ITB1 | 11 | 5.2 | 88.08 |
| | | | FELCA FIBRONECTIN RECEPTOR BETA SUBUNIT | | | |
| | | | PRECURSOR (INTEGRIN BETA-1) (CD29) | | | |
| | | | (INTEGRIN VLA-4 BETA SUBUNIT) | | | |
| 2 | 3.1e−006 | — | gi\|479805\|pir∥S35458 | 12 | 7.0 | 88.59 |
| | | | SNF2 protein homolog - human (fragment) | | | |
| +3 | 7.7e−007 | — | gi\|5725250\|emb\|CAB52406.1\| | 8 | 5.9 | 94.65 |
| | | | (AJ245661) G7 protein [Homo sapiens] | | | |
| | — | — | gi\|3108220\|gb\|AAC62533.1\| | 8 | 6.0 | 92.87 |
| | | | (AF048986) MutS homolog 5 [Homo sapiens] | | | |
| | — | — | gi\|4505253\|ref\|NP 002432.11 mutS | 8 | 6.0 | 92.86 |
| | | | (E. coli) homolog 5 | | | |
| 4 | 6.7e−007 | — | gi\|7512247\|pir∥I65253 | 14 | 6.6 | 80.82 |
| | | | disintegrin-like testicular metalloproteinase (EC | | | |
| | | | 3.4.24.-) IVb - crab-eating macaque (fragment) | | | |
| 5 | 1.8e−007 | — | gi\|10438454\|dbj\|BAB15248.1\| | 19 | 6.4 | 80.60 |
| | | | (AK025824) unnamed protein product | | | |
| | | | [Homo sapiens] | | | |
| 6 | 5.4e−008 | — | gi\|1586344\|prf∥2203411A | 10 | 5.7 | 99.37 |
| | | | reeler gene [Mus musculus] | | | |
| 7 | 3.0e−008 | — | gi\|4503165\|ref\|NP 003581.1\| | 16 | 9.0 | 88.91 |
| | | | cullin 3 | | | |
| +8 | 1.4e−008 | — | gi\|6681275\|ref\|NP 031934.1\| | 14 | 5.2 | 81.72 |
| | | | eukaryotic elongation factor-2 kinase | | | |
| | — | — | gi\|6978795\|ref\|NP 037079.1\| | 9 | 5.1 | 81.47 |
| | | | eukaryotic elongation factor 2 kinase | | | |
| 9 | 1.2e−008 | — | gi\|7662434\|ref\|NP 055733.1\| | 15 | 9.5 | 91.71 |
| | | | KIAA0990 protein | | | |
| 10 | 5.2e−009 | — | gi\|7662436\|ref\|NP 055749.1\| | 13 | 5.8 | 96.68 |
| | | | KIAA0996 protein | | | |

Input Summary

| | |
|---|---|
| Date & Time | Wed Feb 07 10:07:52 2001 UTC (Search Time: 5.88 sec.) |
| Sample ID | EDB Fibronektin, #0824, Bande 7 |
| Database | NCBInr [..\databases\nr] |
| Taxonomy Category | Mammalia (mammals) |
| Protein | 80-100 kDa |
| Mass Range | |
| Protein pl Range | 0.0-14.0 |
| Search for | Single protein only |
| Digest Chemistry | Trypsin |
| Max Missed Cut | 2 |
| Modifications | +C3H5ON@C(Partial); +O@M(Partial); |
| Charge State | MH+ |
| Peptide Masses | |
| (Da, Average) | |
| Tolerance(AVG) | 1.00 ppm |
| | 881.213 983.479 1222.615 1266.561 1376.698 1422.672 1473.821 1479.786 |
| | 1553.850 1567.725 1639.856 1781.886 1819.830 1915.945 1931.961 |
| Peptide Masses | 1961.051 2019.150 2068.101 2224.061 2283.101 2344.093 2470.201 |
| (Da, Monoisotopic) | 2501.215 2705.264 2776.358 2840.545 2872.558 3052.493 3143.494 |
| | 3159.559 3280.571 3298.572 |
| Tolerance(MON) | 50.00 ppm |
| Number of Peptides | 32 |

ProteoMetrics' ProFound is based on ProFound at The Rockefeller
University [search + transmission time: >=5.91 sec]
NOTE:
1. To search again using unmatched masses, click the symbol ®.
2. Highly similar protein sequences were given the same rank (IE user click “+” to expand/contract).

1-25. (canceled)

26. Process for screening compounds that bind to a receptor of the ED_{b}-fibronectin domains, whereby the process comprises:
comparison of a response of cells in the presence of one or more of these compounds with the control response of said cells in the absence of these compounds, whereby the cells express a protein or
a) that has the ability to bind specifically to the ED_{b}-fibronectin domains;
b) that is expressed or activated specifically in endothelial cells;
c) that is expressed or activated specifically in the stromal cells of a tumor;
d) that is expressed or activated specifically in tumor cells;
e) whose binding to the ED_{b}-fibronectin domains is inhibited by a polypeptide; and
f) that has an apparent molecular weight of 120-130 kDa for the light chain and 150-160 kDa for the heavy chain, determined by SDS-polyacrylamide gel electrophoresis.
comprise a nucleic acid that codes for this protein, and whereby the response or the control response is mediated by a receptor of the ED_{b}-fibronectin domains.

27. Process according to claim 26, whereby the response or control response comprises the adherence of cells to surfaces that are coated with the ED_{b}-fibronectin domains or portions thereof.

28. Process according to claim 26, wherein a binding region of the ED_{b}-fibronectin domains comprises sequences SEQ ID NOS: 1-4 or portions thereof.

29. Process according to claim 26, wherein the response or the control response comprises the proliferation of cells on surfaces that are coated with the ED_{b}-fibronectin domains or portions thereof.

30. Process according to claim 26, wherein the response or the control response comprises the proliferation, migration and differentiation of endothelial cells in a collagen matrix that is mixed with the ED_{b}-fibronectin domains or portions thereof.

31. Process according to claim 26, whereby the compounds are selected from the group that comprises antibodies, artificial antibodies, antibody fragments, peptides, low-molecular compounds, aptamers and Spiegelmers.

32. Process according to claim 31, wherein the antibodies are recombinant antibodies.

33. Process according to claim 31, wherein the antibodies are selected from the group that comprises scFv and fragments thereof.

34-55. (canceled)


SEQUENCE LISTING (22 sequences)

SEQ ID NO: 1 - protein, 15 aa, Artificial Sequence
  Description of Artificial Sequence Synthetic peptide
VDITDSSIGLRWTPL

SEQ ID NO: 2 - protein, 15 aa, Artificial Sequence
  Description of Artificial Sequence Synthetic peptide
GYYTVTGLEPGIDYD

SEQ ID NO: 3 - protein, 15 aa, Artificial Sequence
  Description of Artificial Sequence Synthetic peptide
TGLEPGIDYDISVIT

SEQ ID NO: 4 - protein, 91 aa, Artificial Sequence
  Description of Artificial Sequence Synthetic peptide
EVPQLTDLSFVDITDSSIGLRWTPLNSSTIIGYRITVVAAGEGIPIFEDFVDSSVGYYTVTGLEPGIDYDISVITLINGGESAPTTLTQQT

SEQ ID NO: 5 - protein, 4 aa, Artificial Sequence
  Description of Artificial Sequence Synthetic peptide
RGDS

SEQ ID NO: 6 - protein, 15 aa, Artificial Sequence
  Description of Artificial Sequence Synthetic peptide
DTIIFEVPQLTDLSF

SEQ ID NO: 7 - protein, 15 aa, Artificial Sequence
  Description of Artificial Sequence Synthetic peptide
EVPQLTDLSFVDITD

SEQ ID NO: 8 - protein, 15 aa, Artificial Sequence
  Description of Artificial Sequence Synthetic peptide
TDLSFVDITDSSIGL

SEQ ID NO: 9 - protein, 13 aa, Artificial Sequence
  Description of Artificial Sequence Synthetic peptide
ITDSSIGLRWTPL

SEQ ID NO: 10 - protein, 15 aa, Artificial Sequence
  Description of Artificial Sequence Synthetic peptide
SSIGLRWTPLNSSTI

SEQ ID NO: 11 - protein, 15 aa, Artificial Sequence
  Description of Artificial Sequence Synthetic peptide
RWTPLNSSTIIGYRI

SEQ ID NO: 12 - protein, 15 aa, Artificial Sequence
  Description of Artificial Sequence Synthetic peptide
NSSTIIGYRITVVAA

SEQ ID NO: 13 - protein, 15 aa, Artificial Sequence
  Description of Artificial Sequence Synthetic peptide
IGYRITVVAAGEGIP

SEQ ID NO: 14 - protein, 15 aa, Artificial Sequence
  Description of Artificial Sequence Synthetic peptide
TVVAAGEGIPIFEDF

SEQ ID NO: 15 - protein, 15 aa, Artificial Sequence
  Description of Artificial Sequence Synthetic peptide
GEGIPIFEDFVDSSV

SEQ ID NO: 16 - protein, 15 aa, Artificial Sequence
  Description of Artificial Sequence Synthetic peptide
IFEDFVDSSVGYYTV

SEQ ID NO: 17 - protein, 9 aa, Artificial Sequence
  Description of Artificial Sequence Synthetic peptide
FEDFVDSSV

SEQ ID NO: 18 - protein, 15 aa, Artificial Sequence
  Description of Artificial Sequence Synthetic peptide
VDSSVGYYTVTGLEP

SEQ ID NO: 19 - protein, 15 aa, Artificial Sequence
  Description of Artificial Sequence Synthetic peptide
GIDYDISVITLINGG

SEQ ID NO: 20 - protein, 15 aa, Artificial Sequence
  Description of Artificial Sequence Synthetic peptide
ISVITLINGGESAPT

SEQ ID NO: 21 - protein, 15 aa, Artificial Sequence
  Description of Artificial Sequence Synthetic peptide
LINGGESAPTTLTQQ

SEQ ID NO: 22 - protein, 15 aa, Artificial Sequence
  Description of Artificial Sequence Synthetic peptide
ESAPTTLTQQTAVPP